(19) United States
(12) Patent Application Publication
(10) Pub. No.: US 2024/0191891 A1
(43) Pub. Date: Jun. 13, 2024

(54) DISINFECTANT, GAS ACCUMULATION AND COMBUSTION CONTROL DEVICE

(71) Applicant: NanoSieve, Inc., Miami Beach, FL (US)

(72) Inventor: Gabrielle Abizeid, Miami Beach, FL (US)

(21) Appl. No.: 18/403,969

(22) Filed: Jan. 4, 2024

Related U.S. Application Data
(63) Continuation-in-part of application No. 17/885,141, filed on Aug. 10, 2022.
Application No. 17/885,141 is a continuation-in-part of application No. 17/525,848, filed on Nov. 12, 2021.
Application No. 17/525,848 is a continuation-in-part of application No. 16/994,909, filed on Aug. 17, 2020, now Pat. No. 11,473,794.

(51) Int. Cl. F24F 8/10; A61L 9/20; B01D 46/00; B01D 53/04; F24F 11/77; F24F 110/40; F24F 110/65

(52) U.S. Cl. CPC F24F 8/10; A61L 9/205; B01D 46/0028; B01D 46/0036; B01D 53/0446; B01D 53/0454; F24F 11/77; A61L 2209/111; A61L 2209/12; A61L 2209/14; A61L 2209/16; B01D 2257/91; B01D 2257/93; B01D 2259/4508; F24F 2110/40; F24F 2110/65

(57) ABSTRACT

A gas accumulation and combustion control device combining a sorption system, a ventilation system, a control system, and sensor system, with the sensor system configured to detect gas contaminants, transmit a gas detection signal to the control system, the control system configured to adjust the ventilation system based on the gas detection signal, the ventilation system configured to draw the contaminated air in from the atmosphere and lead it toward the sorption system, which in turn is configured to adsorb or absorb the gas contaminants.

### PRIORITY

This application is a continuation-in-part of and claims priority to U.S. non-provisional application Ser. No. 17/885,141, filed Aug. 19, 2022, which in turn is a continuation-in-part of and claims priority to U.S. non-provisional application Ser. No. 17/525,848, filed Nov. 12, 2021, which in turn is a continuation-in-part of and claims priority to U.S. non-provisional application Ser. No. 16/994,909 filed Aug. 17, 2020, now patent U.S. Ser. No. 11/473,794B2, issued Oct. 18, 2022, priority of which is also claimed by the present application. All referenced applications are incorporated herein in their entirety as if restated in full.

### BACKGROUND

The two main dangers of gas accumulation, whether in residential, commercial, laboratory, or industrial settings, include their flammability and their toxicity.

Fires are put out with great difficulty and expense, and cause damage not only to property, which can be extensive, but also to human (and animal) life. A gas fire is exceptionally dangerous, because gas not only burns but may combust, an effect which causes a sudden and massive spread of fire. Since gas is capable of squeezing through cracks or gaps and permeate through different surface, gas may spread from room to room in a manner much faster than traditional fires, which rely on solid media, such as wood. A gas fire is also easier to start than a traditional fire, since gas ignites instantly while solid media such as wood take longer. Further, since gas travels in a near random path, or else are blown about by even low-level currents, gas may enter areas where small fires would otherwise be acceptable due to their controlled nature and distance from more obviously flammable material, such as paper or wood. A person lighting a cigarette or a candle may not realize that they are triggering an explosion because of a stream of gas which has trickled in and accumulated in their room.

While the toxicity of gas generally does not affect property, it can be harmful, even lethal, to living organisms, such as people and animals. Even if a toxic gas is not flammable, the accumulation of gas, which is often undetected, may enter a living being's respiratory and circulatory system, killing otherwise healthy cells, particularly cells in the lungs, esophagus, nasal passage, and brain. Certain gases, such as carbon monoxide, may cause the types of damage described without even requiring a build-up, and such gases are immediately dangerous even in miniscule amounts.

Importantly, flammable and toxic gases are frequently odorless; and when they do have odors, those odors may be very faint. People have varying degrees of sensitivity to odors, and so gases that might be detected by one person may not be detected by another. Even if a person is sensitive to smells, the slow build up of gas may unconciously adjust the person's sensitivity, such that a gas they would otherwise be detected may be undetected if the person has remained in the location during the gas build up.

What is needed is a device that can detect the presence of gas, isolate it through sorption, delay the negative effects of gas build-up through partial and/or continuous sorption, alert a location custodian of its presence, address a max sorption capacity event, and be easy to handle and control. Such a device may nullify the danger for small amounts of gas, or give an attended time to take remedial action, such as opening a window, calling the fire department, and/or evacuating the premises.

### SUMMARY

The gas accumulation and combustion control device comprises a sorption box designed to hold a sorption system, a ventilation system, a sensor system, and a control system. The ventilation system is in electrical communication with the control system, which in turn is in informational communication with the sensor system.

The sorption box is essentially an enclosure against an atmosphere surrounding the sorption box. It has at least one or more passage walls, and one or more pass-through walls, which together form an internal cavity.

The pass-through walls are configured to permit air to flow between the cavity and the atmosphere, and the passage walls, which span from one pass-through wall to the other, is designed to contain the various systems.

The systems are configured to intelligently extract gas contaminants from the environment by actively accelerating air flow into the cavity and then absorbing or adsorbing the gas contaminants by means of sorption material.

### BRIEF DESCRIPTION OF THE DRAWINGS

FIG. 1 shows a gas accumulation and combustion control device.

FIG. 2 shows a sorption box with pass-through walls.

FIG. 3 shows the ventilation system, control system, and sorption system being disposed inside inlet and outlet doors.

FIG. 4 shows sorption chambers and openings.

FIG. 5A-C show various fan configurations.

FIG. 6 is a flowchart showing conversions between fan configurations.

FIG. 7A-C shows the sensor system.

FIGS. 8-9 are flowcharts showing system processes.

FIG. 10 shows sensor system configurations.

FIG. 11-13 are flowcharts showing system processes.

FIG. 14 show various configurations of multiple gas accumulation and combustion control devices.

FIG. 15 shows a number of equations that may be used to calculate the volume of the sorption box.

FIG. 16 shows sorption units stacked cylindrically.

FIG. 17 shows sorption units stacked cylindrically.

FIG. 18 shows a sorption unit coupled to an ultra violet light emitter.

FIG. 19 shows a the device featuring collection containers.

FIG. 20a shows the collection container in a compressed or non-expanded state.

FIG. 20b shows the collection container in an expanded state.

FIG. 21 is a flowchart showing an alternating sorption box desorption system process.

FIG. 22 is a flowchart showing a vetilation engagement system process.

FIG. 23 is a flowchart showing a transmit signal system process.

### DETAILED DESCRIPTION

The gas accumulation and combustion control device is designed to prevent the accumulation of flammable and toxic gases in a residentical, commercial, laboratory, or industrial setting.

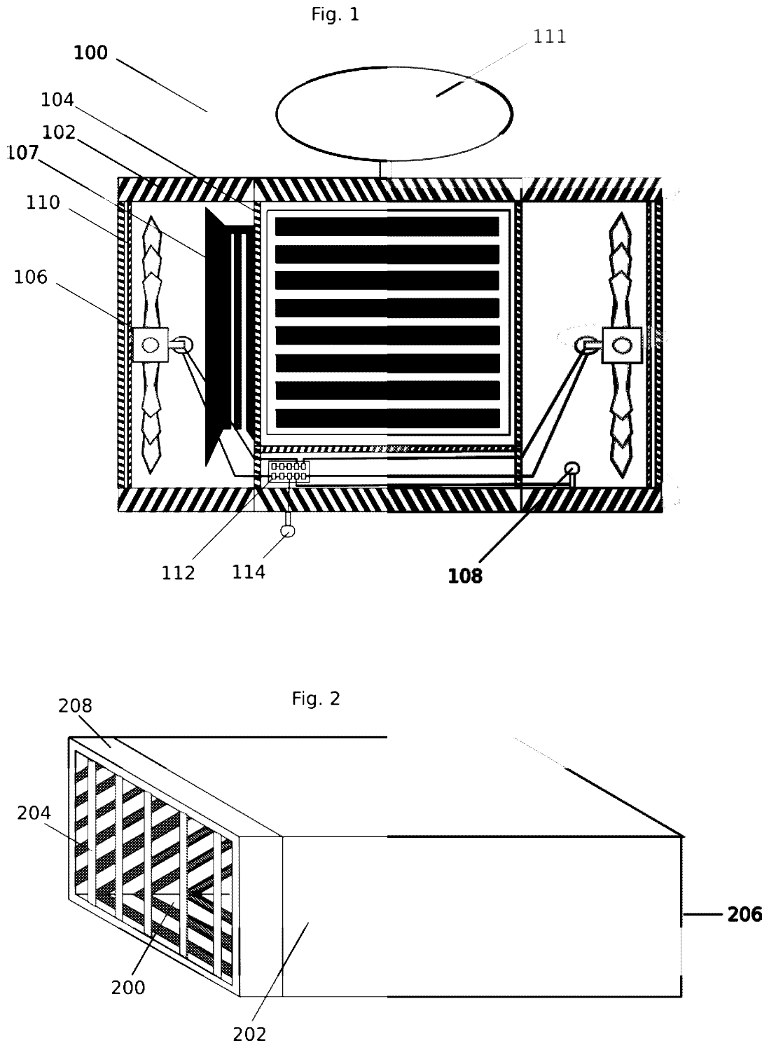

As shown in FIG. 1, the gas accumulation and combustion control device 100 comprises a sorption box 102, a sorption system 104, a ventilation system 106, a compressor 107, a pressure regulator 108, a dust filter 110, a control system 112, a gas collection container 111, and a sensor system 114. These components may be attached to each other mechanically, electrically, wirelessly, directly, and/or indirectly. The attachment may be permanent, temporary, removable, or replaceable.

The sorption box is an enclosure, preferably made of metal, such as aluminum or steel, a hard plastic, or a combination thereof. As shown in FIG. 2, the sorption box may be rectangular or tubular in shape and comprise a cavity 200 surrounded by one or more passage walls 202, and two pass-through walls, including one or more inlet walls 204 and one or more outlet walls 206, with the inlet and outlet walls configured to provide an inlet into and an outlet from the passage walls, respectively. The inlet and outlet walls may be grates in which air, particularly air mixed with flammable or toxic gases, and other small particles are permitted through while preventing entrance into the cavity by larger particles, such as those greater than 1 mm in diameter. In one variation, the inlet and outlet walls may each be coupled to inlet and outlet doors 208, respectively. The inlet and outlet doors may each comprise a set of shutters 210, the shutters oriented to permit fluid flow when in a substantially orthogonal orientation, to block fluid flow when in an orientation substantially in line with the doors, and to permit limited fluid flow when in an orientation between open and closed. The orientation may be controlled by the control system, which will be described below. The inlet and outlet doors may be slidably or hingedly attached to the inlet and outlet walls and configured to seal the inlet and outlet walls.

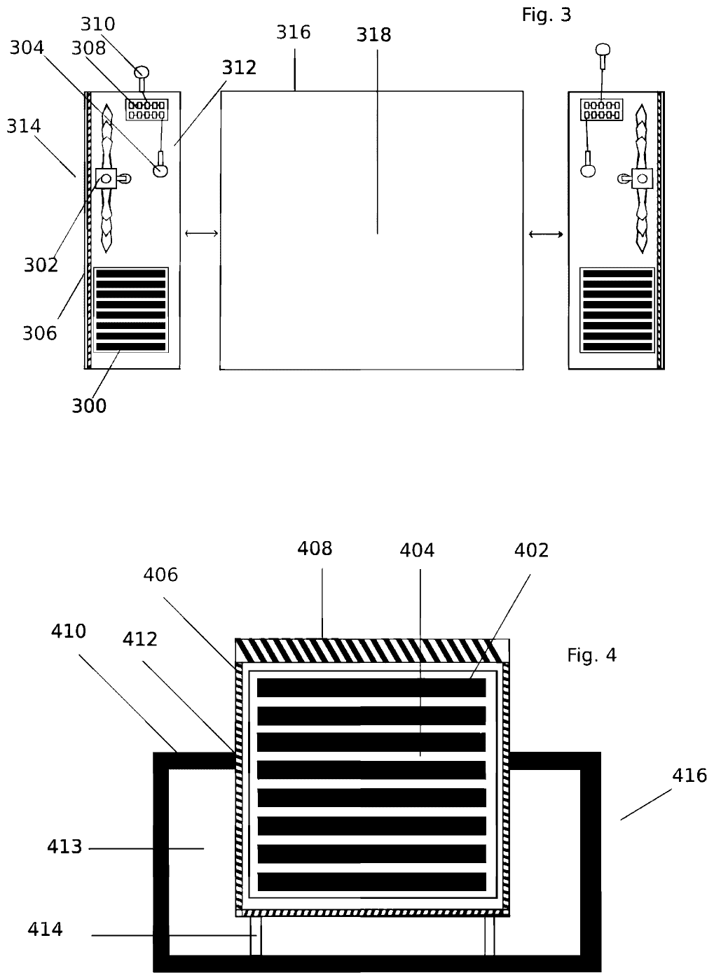

In one variation, as shown in FIG. 3 the sorption system 300, ventilation system 302, pressure regulator 304, dust filter 306, control system 308, and sensor system 310 may be disposed within the interior 312 or on the surface of the inlet and/or outlet doors 314, with the doors being removably disposed in the sorption box 316. When the inlet and/or outlet doors are removed from the sorption box, the sorption box may be thereafter operate as simply an additional pipe or vent section in a larger HVAC system.

The sorption box may be configured to connect adaptably to tubing, piping, vents, or other HVAC components. The sorption box may be built into new HVAC systems or retrofitted into existing systems. It may be screwed or nailed in, or otherwise locked into place. The inlet and/or outlet walls may feature mechanisms, such as latch or screw-fit components, to adapt to the HVAC components. The sorption box may be positioned such that it is substantially or at least partly inside a building with the outlet wall positioned outside the building. Alternatively, the sorption box may be located inside a room in which filtering and adsortion is desired, or behind the wall of such a room but with access thereto. In one variation, the sorption box is independent of other HVAC components but is instead a stand-alone machine. As shown in FIG. 4, the sorption system may feature sorption units 402, the sorption units capable of adsorbing or absorbing flammable and/or toxic gases. The sorption units may be pads or packs made of or filled with sorption material. The sorption material may also be provided in coils, particularly meshed coils, thereby increasing the surface area of sorption. The sorption material may substantially fill the sorption box cavity, or, In order to facilitate replacement, the sorption units may be placed in and removed from sorption chambers 404, which are disposed inside the cavity. The chambers may hold the sorption units in place while still permitting airflow thereupon. A chamber may consist at least in part of a cage 406, which would enable air to enter while preventing a sorption unit from falling out. The cage may consist of wire or bars arranged latitudinally, longitudinally, diagonally, or in any other appropriate pattern. The cage may also comprise a mesh or floating screen.

The chambers may feature hatches 408 which provide access to the sorption units from outside the sorption box, but are also capable of being closed in order to prevent access thereof. The hatches may be substantially continuous and in line with the passage walls 410, being hingedly or slidably attached and engaged to the stationary portion of the passage walls.

In one variation, the sorption chambers themselves may be removable from the sorption box. The chambers may be fitted into chamber openings 412 that are disposed in the passage walls of the sorption box. The chambers and chamber openings may be screw-fit, constructed so that the former fits tightly into the latter, or otherwise configured to prevent the chambers from falling out of the chamber openings due to gravity or other unintended forces without grossly impeding a user from removing them. The chambers themselves may be disposed on a track 414 disposed inside the cavity and slidably removable from the sorption box 416.

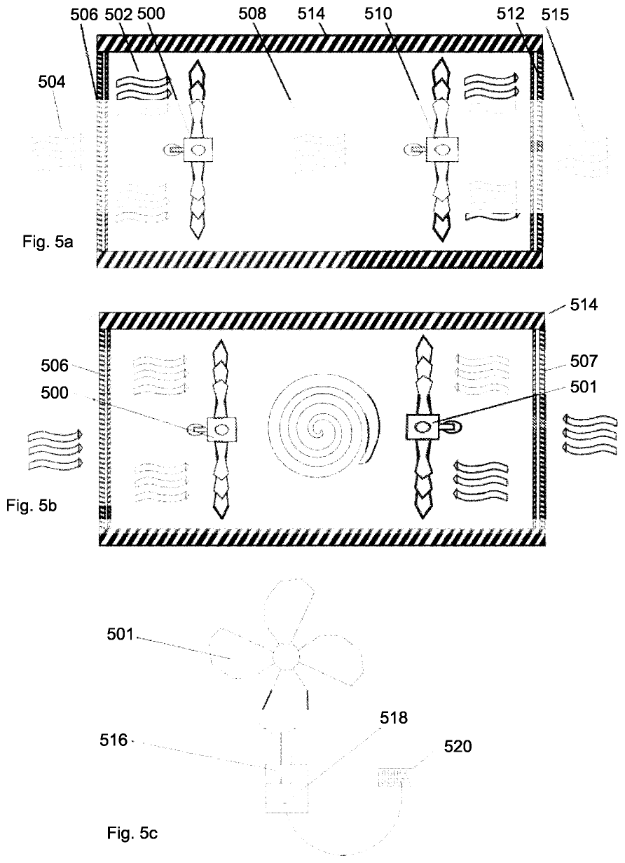

In one embodiment, the ventilation system may comprise an inlet fan and an outlet fan, with the inlet fan positioned close to the inlet wall and the outlet fan being positioned next to the outlet wall. The fans have a diameter approximating the sorption box diameter, so that all air entering the inlet wall may encounter and be handled by the inlet fan, and all air passing through the cavity may encounter and be handled by the outlet fan. As shown in FIG. 5A, the fans may be oriented so that the inlet fan 500 sucks in air 502 from the atmosphere 504 outside the inlet wall 506 and blows the air toward the cavity 508; conversely, the outlet fan 510 sucks in air from the cavity and blows the air toward and through the outlet wall 512 and out of the sorption box 515 However, in a preferred embodiment, as shown in FIG. 5B, the outlet fan operates as a second inlet fan, such that both fans suck air from the environment and blow air into the cavity. In this preferred embodiment, the sorption box 514 consists of a first and second inlet wall 506, 507 and a first and second inlet fan 500, 501, with the first inlet fan disposed behind the first inlet wall and the second inlet fan disposed behind the second inlet wall. In lieu of or in combination with the fans may be a ventilation assembly additionally comprising a compressor/blower and/or vacuum pump.

In the preferred embodiment described above, as shown in FIG. 5c, the second inlet fan may be disposed on a rotation mechanism 516, such as a rotating platform or a rotating axle, which enables the second inlet fan to rotate from a cavity-facing orientation to an atmosphere-facing orientation, thereby converting the sorption box from containment-type chamber to pass-through type. The rotating mechanism may be controlled manually by a user, such that the rotating mechanism may be physically rotated directly or indirectly by the user, or electrically. The rotating mechanism may be connected to a motor 518 which, when receiving a (wired or wireless) signal from the control system 520, a dedicated module, or a mobile device, will cause the rotating mechanism to rotate and thereby change the second inlet fan's orientation.

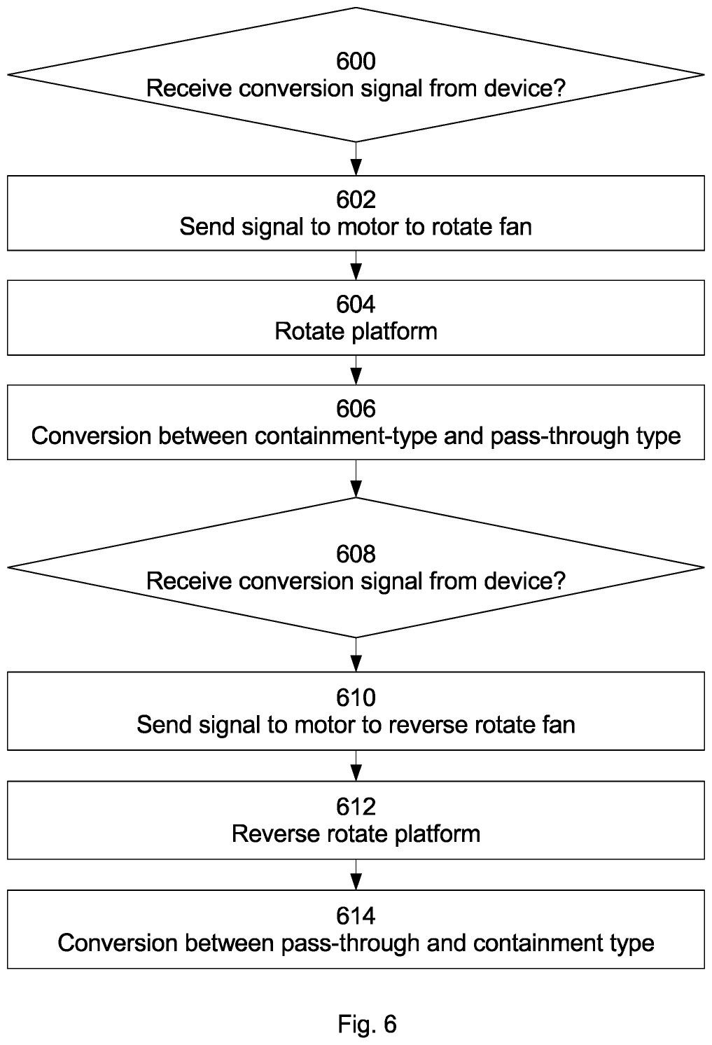

The conversion between containment-type and pass-through type, as shown in FIG. 6, may work as follows: The control system may receive a signal from a mobile device 600 to convert the containment-type chamber to a pass-through type chamber. The control system may send a signal to the motor to rotate the second inlet fan 602. The motor will then operate, causing the platform on which the second inlet fan resides to rotate 604. The inlet fan will then experience a full rotation (of approximately 180 degrees) 606, and conversion between the containment-type chamber and the pass-through type chamber will be achieved. If the control system receives a second signal from the mobile device 608 to convert the pass-through type chamber to a containment-type chamber, the control system will send a signal to the motor to reverse rotate the second inlet fan 610. The motor will then reverse rotate the second inlet fan 612 and the inlet fan will experience a full rotation 614, and conversion between the pass-through type chamber and the containment-type chamber will be achieved. Conversion may also be effected by reversing the spin direction of the fan(s).

The compressor may be disposed between the inlet fan and/or door and the cavity, and configured to reduce the volume of the gas in order to facilitate sorption by the sorption units. An additional or alternative compressor, optionally coupled to a vacuum pump, may be disposed at the outlet of the sorption chamber; this configuration enables the desorption, reuse, and replenishment of the sorption material.

The gas collection container may be rigid or made of inflatable material. It is preferably in fluid communication with the cavity, thereby leeching densified and contaminated air from the sorption box. This gas collection container may, in one variation, be intermediated by a ventilation fan in order to accelerate gas collection.

Transport of contaminated or cleaned air may be facilitated by a series of valves intermediating the various components of the device. For example, a first set of valves may control flow from the compressor to the cavity, a second set of valves may control flow from the cavity to the gas collection container, and a third set of valves may control flow from the cavity to outlet fans or to the outlet wall.

The dust filter is (dust filters are) preferably disposed within or behind the inlet wall(s). The dust filter is configured to catch particles smaller than 1 mm in diameter which the inlet wall(s) otherwise might not catch, such as dust particles, which are between 2.5 and 10 microns.

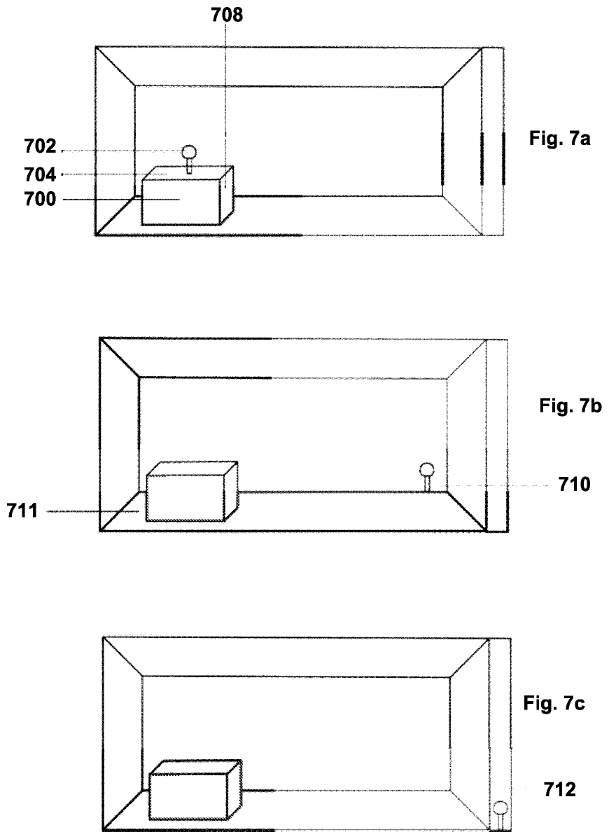

As shown in FIGS. 7A-C, the sensor system 702 may be positioned anywhere on the sorption box 700, such as the passage 704, inlet or outlet walls 708, or elsewhere, such as in one part of a room 710 while the sorption box is located in another part of the room 711 or even within a wall 712. If the sensor system is not physically attached to the sorption box, it may be connected via wires or engaged to the control system via a wireless interface, such as bluetooth or WiFi.

The sensor system may include a gas sensor configured to detect flammable or toxic gases. Examples of gas sensors include metal oxide based gas sensor, optical gas sensor, electrochemical gas sensor, capacitance-based gas sensor, calorimetric gas sensor, or acoustic based gas sensor. The gas sensor may consist of sensing elements such as a gas sensing layer, a heater coil, an electrode line, a tubular ceramic, or an electrode. Examples of gases which may be sensed include methane, butane, LPG, smoke, alcohol, ethanol, CNG gas, natural gas, carbon monoxide, carbon dioxide, nitrogen oxides, chlorine, hydrogen gas, ozone, hydrogen sulfide, ammonia, benzene, toluene, propane, formaldehyde, and other various toxic or flammable gases.

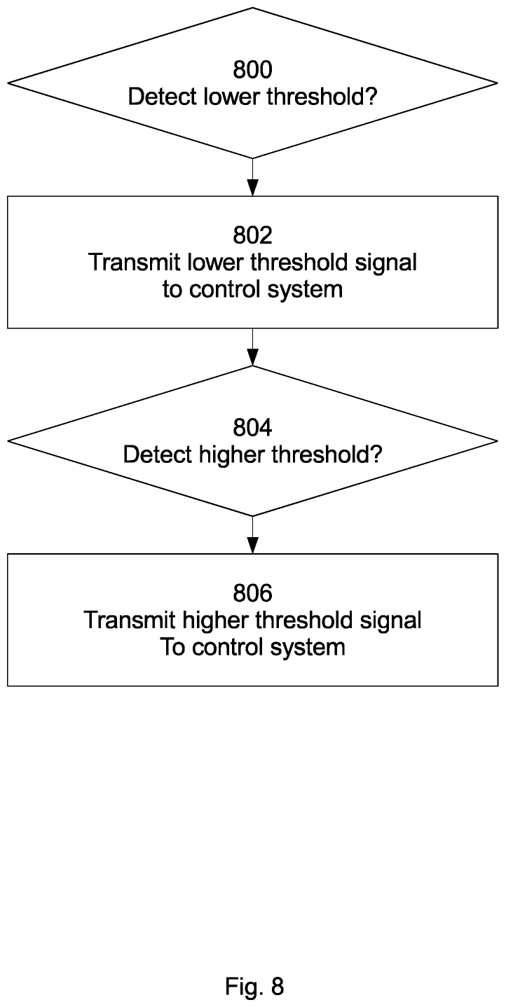

Upon detecting a designated concentration level of an undesirable gas, the sensor system is configured to transmit a gas detection signal to a wireless receiver inside the control system. The designated concentration levels of undesirable gases may be based on lower flammability limits or on recognized toxicity levels, which are levels where the gas becomes dangerous to human or animal health. In one variation, as shown in FIG. 8, there are two separate gas detection levels—a lower threshhold and an upper threshhold. In this variation, the sensor system transmits a lower threshhold gas detection signal to the wireless receiver 802 upon detecting a lower threshhold of gas 800, which is a level which it is considered advisable to human operators or users but which does not yet reach or approach the lower flammability limits or recognized toxicity levels, and transmits an upper threshhold 806 gas detection signal upon detecting an upper threshhold of gas 804.

The sensor system may be configured to detect the concentration of a given gas, approximate that concentration numerically, and transmit the numerical concentration to the control system or directly to a visual display to enable users or operators to view and track the gas levels. The concentration levels may be captured and transmitted in real time, or captured at reoccurring intervals, such as once an hour, once a day, or once a week. The captured concentration levels may be saved in a database for future reference. In one variation, the concentration levels are transmitted to a dedicated module or mobile device, where they are converted into trending data, and the trending data may be saved on the module or device and displayed upon request by the user.

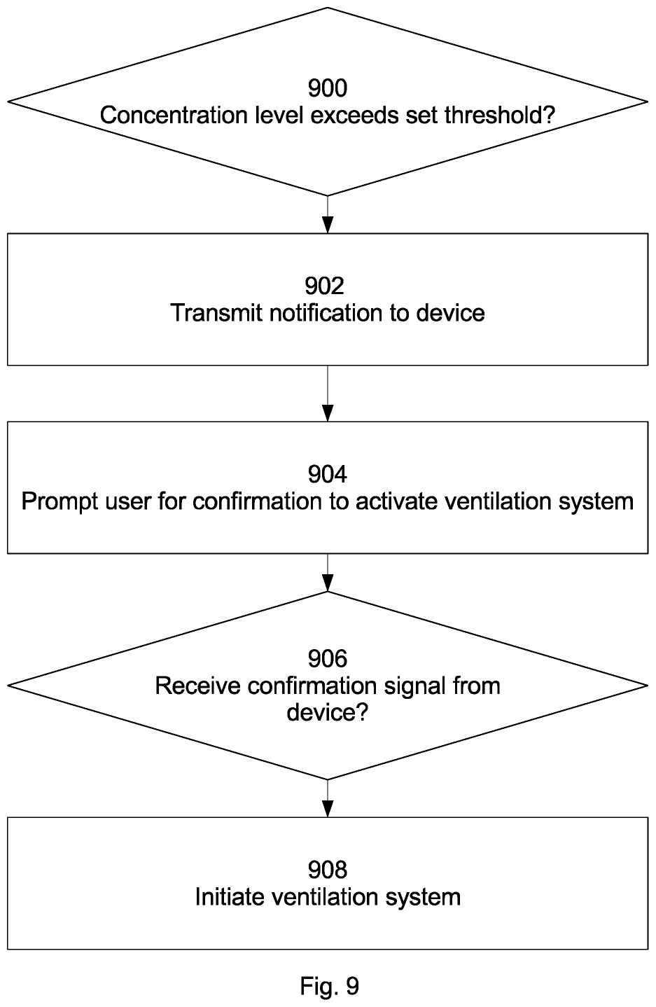

As shown in FIG. 9, upon capturing a concentration level that equals or exceeds a set threshhold 900, the sensor system may transmit a notification wirelessly to the user's mobile device or a dedicated module 902, informing the user of the concentration level or that the concentration level has exceeded the set threshhold, and then prompt the user for confirmation to activate the ventilation system 904. In one version, the ventilation system is automatically activated without requiring user confirmation.

The control system comprises a set of processors and wireless receivers disposed within a container. Upon receiving the wireless detection signal from the mobile device 906, the control system is configured to initiate or permit an electric flow to the ventilation system 908, thereby turning on the fans. In the variation described above, the control system may permit electric flow to the ventilation system upon receiving an upper threshhold gas detection signal, but only turn on a warning signal upon receiving a lower threshhold gas detection signal. The warning signal may be a light, such as a bulb, LED, or other illumination component, configured to illuminate in either a steady stream or flashing pattern, and which is signalled electrically or wirelessly by the control system. The warning signal may be a text message or other notification sent to a human user or operator's phone or a separate display screen. The warning signal may also be an audio transmission, such as a beeping sound, emitted from a speaker disposed on or in the sorption box or else positioned in the targeted room and wirelessly connected to the control system. An exemplary manifestation of the control system may be a SCADA (supervisory control and data acquisition) system, which includes software and hardware elements enabling the control of processes locally or remotely, the monitoring, gathering, and processing of real-time data, interaction with devices such as sensors, valves, pumps, and motors though a human-machine interface, and the recording of events into a log file.

In one variation, the user may communicate with the control system and/or sensor system using the dedicated module or mobile device via a dedicated user interface. The user may observe the concentration levels in real time and observe historical concentration data. The user may send a signal to the control system to turn on the ventilation system based on target concentration levels, which may be set by the user using the user interface, and/or manually.

The control system and/or the ventilation system may be mechanically, hydraulically, or battery operated, feature a plug for inserting into an electrical outlet, and/or hardwired into a building's electrical wiring. If the control system is battery operated, the battery may be contained in a battery box, with the battery box being disposed inside or adjacent to the control system. The battery box may be positioned so that it is accessible from outside the sorption box so that the battery may be easily removed and replaced. The battery box may feature a port which passes through the walls of the sorption box and configured to receive a battery charger.

The control system may impose various activity programs on the components of the device, principally by controlling the electrical flow to the one or more fans and the one or more motors, thereby turning the one or more fans on or off, increasing or decreasing rotations speeds of the one or more fans, or switching the directional orientation between the outlet orientation and the inlet orientation. The control system may also control the valves that permit or block fluid flow from entering the device, moving throughout the device, (such as between the compressor and the cavity, the cavity and the gas collection container, the cavity and the outlet fans), and exiting the device. The doors comprise a row of shutters, such that when the shutters are oriented perpendicular to a door, the door is in an open state, and when the shutters are oriented substantially in line with the door, the door is in a closed state. The shutters may be electrically and mechanically controlled by the control system as well. The control system may additionally control vacuum pumps, blowers, compressors, and any and all other electrical parts forming the ventilation system.

In one program, the control system determines if the sorption units have reached capacity based on the internal contaminant gas signals, and if so, imposes a containment program on the ventilation system, with the containment program featuring either all of the one or more fans turned off or turned on and put into the inlet orientation. The containment program may be subceeded by a collection program, in which the valves connecting the cavity to the gas collection containers are opened for a span of time, ideally until the gas collection containers are filled to capacity, hereafter the valves are shut off. To assist in determining whether the gas collection containers are filled to capacity, a pressure sensor in signal communication with the control system may be disposed between the valve and the gas collection container. This gas collection container may be removably attached to the cavity such that once it is removed, it may be sealed up. In one variation, the valve is principally attached to the gas collection container and is removed with it. In another variation, the valve is principally attached to the cavity, and the gas collection container must be sealed by other means, such as via a cap or a separate valve.

In another program, the control system determines if the contaminant gas levels in the atmosphere are too high (although this may also be the default assumption for the control system, and therefore a default program). If so, the control system imposes a concentration program on the ventilation system, with the concentration program set for increasing the speed of the one or more fans in an inlet orientation or switching one or more fans from an outlet orientation to an inlet orientation.

In yet another program, the control system determines if the sorption box pressure is too high, and if so, imposes a pass-through program on the ventilation system, with the pass-through program featuring at least one fan in an outlet orientation, cessation of compressors/blowers at the inlet, an adjustment of needle valves/pressure valves throughout the system.

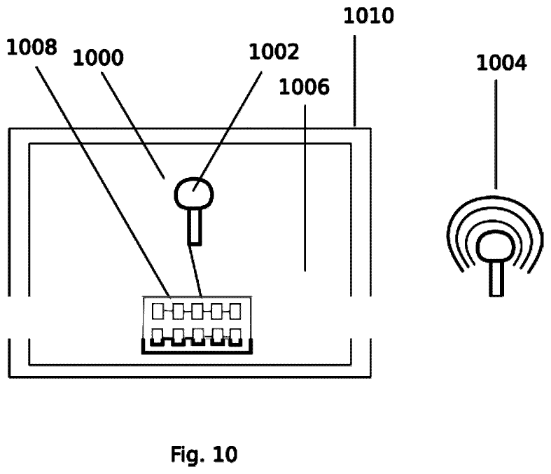

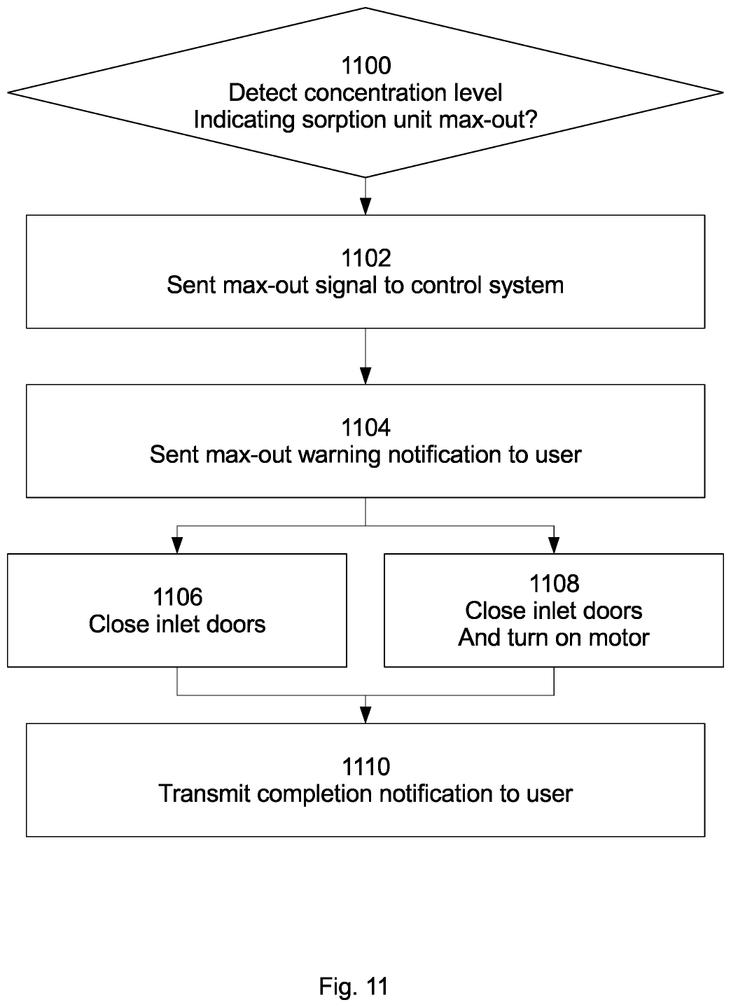

In one variation, as shown in FIG. 10, the sensory system 1000 comprises a first 1002 and second sensory set 1004, with the first sensory set being positioned inside the cavity 1006 and being electrically connected to the control system 1008 and the second sensory set being positions outside the sorption box 1010, perhaps several to many feet away, and connected to the control system using a wireless protocol. This first sensory set may also be in wireless communication with the user's mobile device or dedicated module, and may inform the user when the concentration level of gas in the sorption box is such that the sorption units may have reached their sorption limits, thereby informing the user that the sorption units may need to be checked or replaced. This information may also influence the user, and additional information may be sent suggesting, to close the inlet (and outlet doors), to thereby keep the gas from escaping the sorption box and thereafter turn off the ventilation system, or to rotate the second inlet fan into an outlet fan in order to blow the gas out of the sorption box and into collection bags or an atmosphere outside the building (or into a collecting pipe or apparatus). These steps may also be automated according to the following processes, as shown in FIG. 11: 1. The second sensory set detects a concentration level which indicates that the sorption capacity of the sorption units have maxed out 1100. 2. The control system receives this signal 1102, and then wirelessly transmits a notification to the user 1104. 3a. The control system may trigger the inlet doors to close, and thereby prevent the gas from deadsorbing or deabsorbing and exiting the sorption box 1106, or 3b. The control system may trigger the first inlet door to close and trigger the motor connected to the second inlet fan to rotate into an outlet fan and blow the lingering gases into a collection bag, pipe, or apparatus or into the atmosphere 1108. 4. Transmit a process completion signal to the user 1110. As an additional or alternate step to any of the above, the control system may trigger the inlet valves to close so that the vacuum pump can remove the air trapped within the sorbtion material, thereby conditioning the sorption material for reuse. The control system may trigger valves disposed between the vacuum pump and the outlet to open, thereby enabling the vacuum pump to suck the gas out.

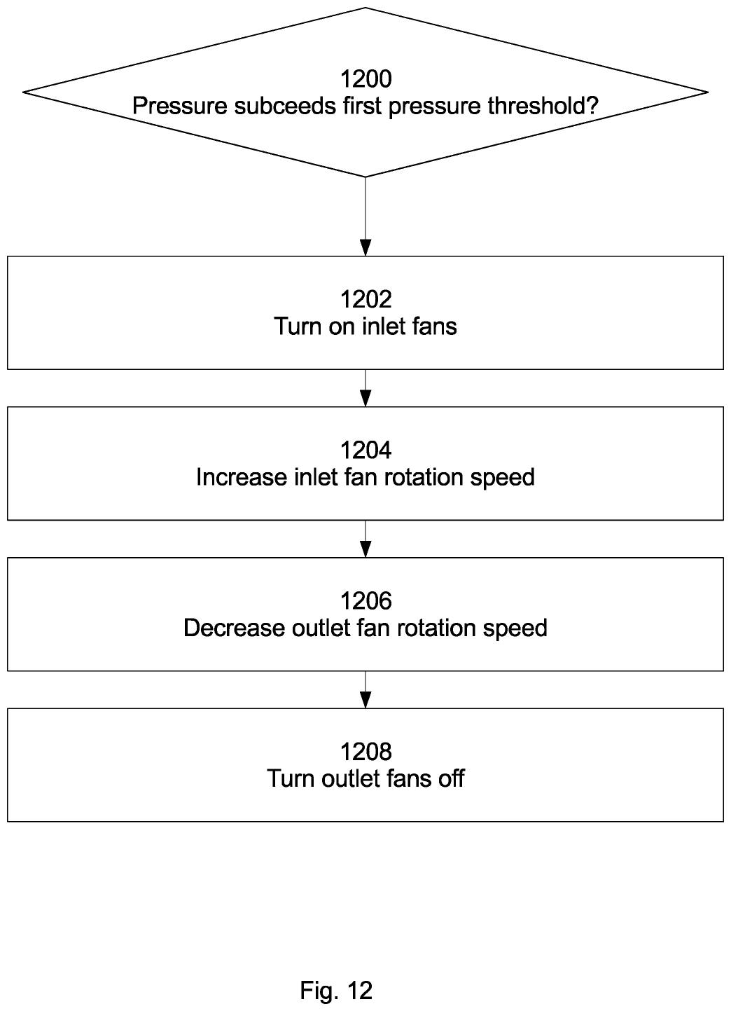

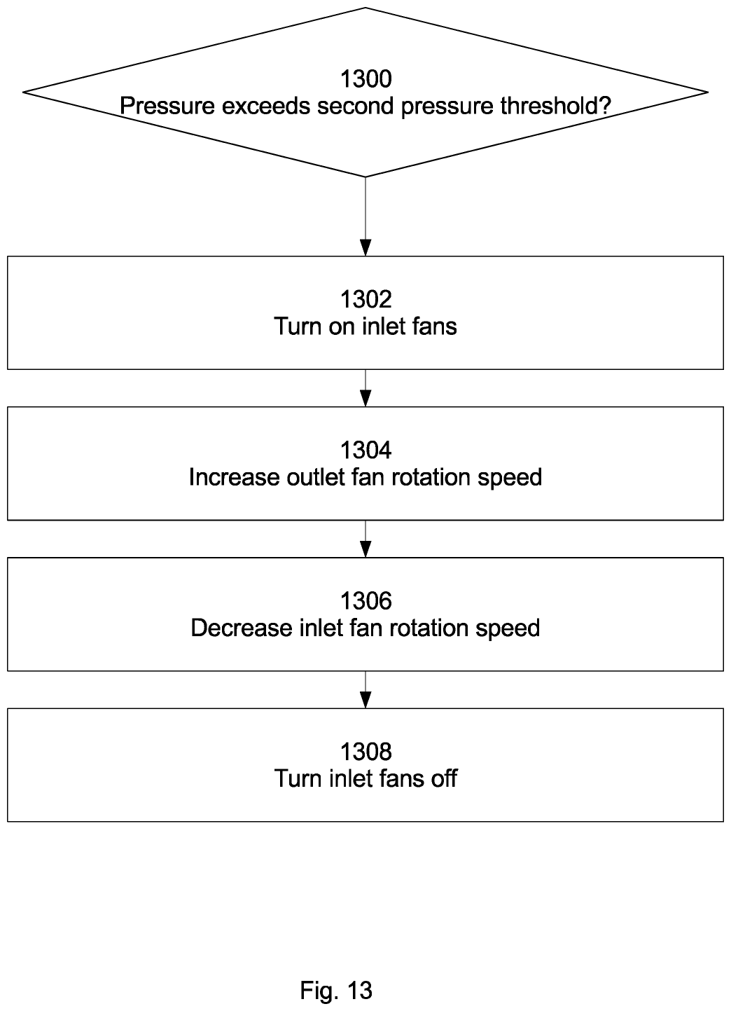

The pressure regulator features a pressure sensor designed to detect the measurement of gas pressure. Based on the degree of pressure imposed on the sensor, the pressure regulator generates an electrical signal to convey the pressure measurement to other components. As shown in FIG. 12, while the pressure subceeds a first pressure threshhold 1200, the pressure regulator may communicate with the ventilation system to turn the input fan(s), compressors/blowers, and vacuum pumps on 1202, permit or instruct the input fan(s) to increase their rotation speed 1204, turn the output fan(s) off 1206, and/or permit or instruct the output fan(s) to decrease their rotation speed 1208, thereby increasing the density of air, and thus the pressure, in the adosorption box. Conversely, as shown in FIG. 13, when the pressure exceeds a second pressure threshhold 1300, the pressure regulator may communicate with the ventilation system to turn the output fan(s) on 1302, permit or instruct the output fan(s) to increase their rotation speed 1304, turn the input fan(s) off 1306, and/or permit or instruct the input fan(s) to decrease their rotation speed 1308, thereby decreasing the density of air, and thus the pressure, in the adosorption box.

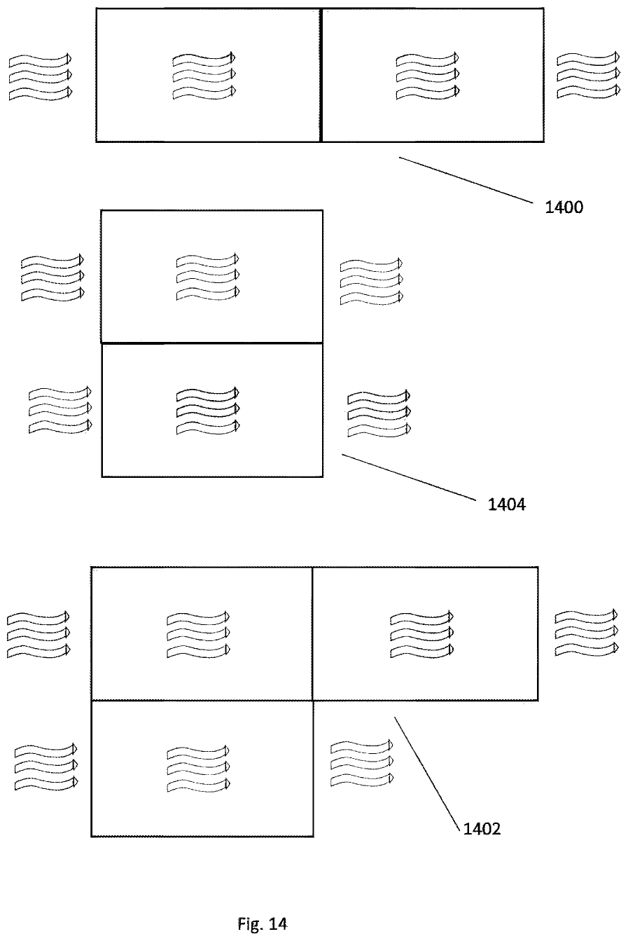

As shown in FIG. 14, a plurality of sorption boxes may be positioned in a series 1400, such that once the sorption capacity of the sorption units of a first sorption box are maxed out, the first sorption box may blow its gas-heavy air into the second sorption box, and so on. A plurality of sorption boxes may simultaneously 1402 or alternately 1404 be positioned in parallel, such that each sorption box has an inlet adjacent to the atmosphere and not to the outlet of any other sorption box. Each sorption box may be dedicated to capturing a different type or category of gas; for example, a first set of sorption boxes may be dedicated and configured to capturing flammable gasses while a second set of sorption boxes may be dedicated and configured to capturing toxic gasses. Each sorption box of the first set may be dedicated to a subset of flammable gasses and each sorption box of the second set may be dedicated to a subset of toxic gasses.

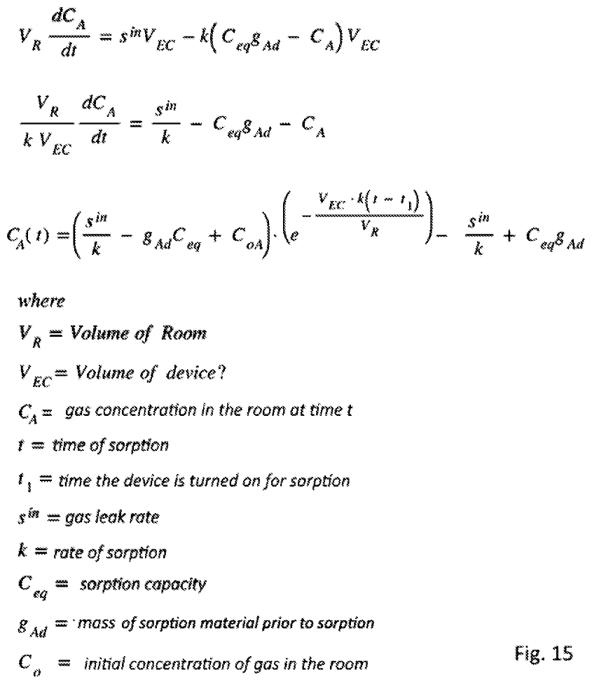

The sorption box may be sized proportional to the space in which filtering and gas sorption is sought, and may be calculated according to the equations shown in FIG. 15.

Additional examples of sorbents include catalytic sorbents, photocatalysts, polymerics, MOFS, Alkali metals such as carbonates and oxides, amine solid sorbents, carbonaceous materials such as carbon nanotubes and carbon molecular sieves, zeolites, mesoporous silica, alumina, hydrotalcite-like compounds (HTICs), metal-based oxides such as CaO based sorbents, porous MgO, Sodium Zirconate, Lithium compounds, and Na₂O promoted alumina, activated carbons, sorbents. So-called photocatalysts, such as titanium dioxide, work to disinfect by, upon being disposed to light, generate hydroxyl radicals.

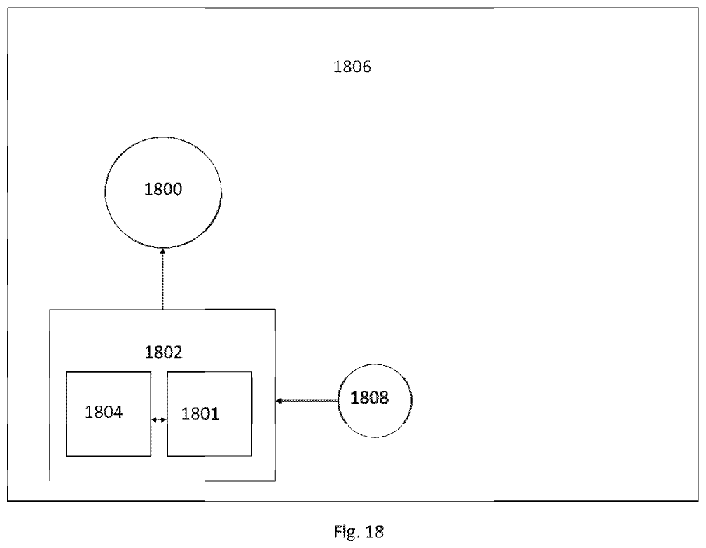

In one embodiment, one or more sorbents and/or the sorption box are coated with crystalline coating material, which is configured to generated hydroxyl radicals upon being exposed to light. Hydroxyl radicals are observed to denature viruses, such as SARS-Coronavirus, by damaging viral exterior features, such as the crown or spike proteins, puncturing the lipid membrane, and exposing the RNA contents. The crystalline coating material may include metal organic frameworks (MOF), which operate as desiccants by providing an enlarged, porous, surface area with external-facing molecules in a cage-like structure that are likely to bind and thereby capture free-floating molecules. The crystalline coating material may be added to traditional sorbents as an applied layer or may be used as sorbents by themselves. The use of crystalline coating material in conjunction with other sorbents and/or the sorption box may also provide disinfecting effects on bacterial and fungal growth. In another embodiment, the sorption box is coupled with an Ultra Violet (UV) emitting bulb or light source. The use of UV is an effective method of denaturing viruses, and acts to damage the exterior features of the virus, thereby exposing and further damaging the RNA contents. As shown in FIG. 18, the UV light source 1800 may be connected electrically and informationally to the sorption box control system 1801 of the sorption box 1802 in order to enable automatic or manual control over the duration, intensity, and wavelength of the UV light. The control system may set the UV light settings based on feedback from the sensor system 1804, which may detect the number of individuals inside a room 1806 using infrared sensors 1808, and whether individuals sneeze or cough using microphones or other sound sensors. In a more advanced feature, the sensor system includes a swab arm that mechanically ushers potential viral matter that has settled on a surface interior or exterior to the sorption box, depending on the sensor setup, into a miniaturized PCR (polymerase chain reaction) or LFT (lateral flow test) testing system, which may include a thermocycler, immunoassay technology using nitrocellulose membranes, colored nanoparticles or labels, and antibodies. After running a PCR or LFT test, the control system can be informed whether viral matter is present, as well as the specific type of viral matter, and run the UV light parameters according to the specific parameters determined to most capably destroy the viral matter.

In one embodiment, the sorption box features a heating mechanism, such as conventional heating elements found in portable heaters, and which are electrically connected to the control system. The control system may provide for manual control over heating, automatic control based on feedback provided by thermometrical sensors, or a combination of the two, such that a user can program the heating elements to activate upon the detection of a lower threshold temperature and deactivate upon the detection of a higher threshold temperature. The user may also program the control system to activate the heating elements based on sorbent activation or dehydration requirements. The heating system may also be used for humidity control in order to maintain the efficacy of the sorption units. Dehumidification may be scheduled or programmed to occur upon the detection of a set humidity threshold. Finally, the control system may be configured to apply a desorption program upon detecting an adsorption saturation point has been reached or based on a schedule.

In one embodiment, the sorption units may feature a multi-sorbent complex, featuring multiple layers stacked together, with each layer comprising a different material, thickness, density, or configuration of sorbents. The layers may be stacked in a pile, or radially such that a first layer comprises a core which is then surrounded nearly entirely by a second layer, and so on.

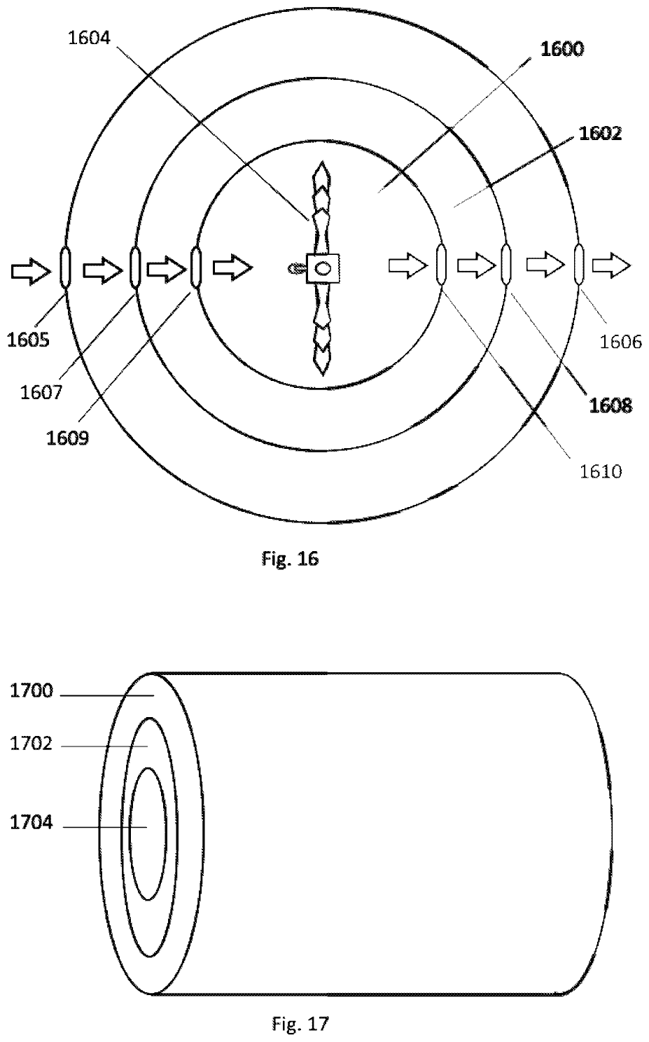

In another embodiment as shown in FIG. 16-17, the sorption boxes may be shaped cylindrically and sized so that one sorption box 1600 may be placed internally to a second sorption box 1602 as an internal cylindrical layer. Each sorption box may have its own dedicated ventilation system so that the sorption boxes may be separated and used independently. In one variation, they may share the mechanical aspects of a single ventilation system 1604 placed in a “core” or inner-most layer and/or the outer-most layer. The ingresses 1605, 1607, 1609 and egresses 1606, 1608, 1610 of the sorption boxes may line up so as to enable the atmosphere to flow through (and be sucked in via the ventilation system) the outer-most layer to the inner-most layer. The sorption boxes may utilize separate and decicated valves, piping, compressors/blowers, and vacuum pumps at each egress, or may share a common set at the final egress. As shown in FIG. 17, the ingresses 1700, 1701, 1703 and egresses (not shown) may also be disposed on the exposed face of all layers so that ingress and egress of atmospheric air may occur directly through each layer without having to first pass through a separate layer. As mentioned previously, each sorption unit may be dedicated to a specific type of gas control and may be activated independently based on the detection of its target gas/contaminant (i.e., dust, pollen, smoke, viral particle, toxic gas, flammable gas, etc.). Thus, a first sorption unit may target toxic gasses, a second sorption unit may target combustible gasses, a third sorption unit may target viral, bacterial, or fungal particles, and a fourth sorption unit may target air purification.

As previously discussed, the control system may transmit a communication to a user's mobile device or to a dedicated device conveying sorption system activity, including instructions to replace one or more sorption units. The control system may also be configured to transmit communications to third parties such as fire departments. The transmission may be wirelessly via Bluetooth, WiFi, or some other wireless protocol. In one embodiment, each sorption box and/or sorption unit is equipped with a scale or other weight measuring mechanism to determine when the sorption unit has reached its saturation point. The system may also make this determination based on measurements of inlet flowrate, concentration, and/or operation/remediation time. The system may also include a user interface configured to inform the user as to the location of sorption unit disposal or recycling services. Such information may be displayed as pins on a map. The system may either itself comprise or be coupled to a GPS application.

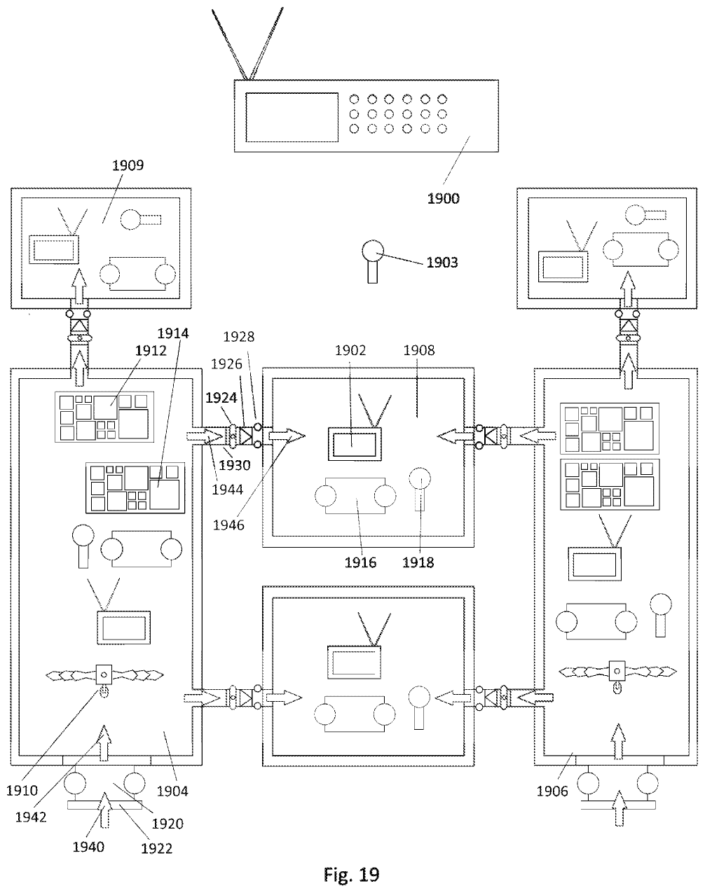

In one embodiment, as shown in FIG. 19, the sorption boxes 1904, 1906 are arranged in parallel, such that each of the sorption boxes are in fluid communication with the ambient air, particularly enabling air flow from the ambient air into the sorption boxes. The sorption boxes are principally configured to absorb or adsorb flammable and/or toxic gasses, and as such, contain various sorption units, including gas sorbents 1912 and humidity sorbents 1914, the latter of which permits more efficient use of the capacity of the gas sorbents. A humidity filter 1922 may also be fitted at the sorption box inlets to decrease the humidity entering in the first instance. Finally, the sorption boxes may be equipped with ventilation systems 1910 in order to impel air inward.

The boxes are each in turn in fluid communication with one or more shared collection containers 1908, and one or more dedicated collection containers 1909, via pipe or tube outlets 1930 fitted with a series of outlet valves 1924, outlet pumps 1926, and outlet compressors 1928. Resident compressors 1916, positioned not at the outlets but within the sorption boxes and collection containers themselves, may be used to further increase the volumetric efficiency of the system. Inlet compressors 1920, positioned at the inlet of the pass-through walls of the sorption boxes, may also be used to compress the air before it enters the sorption boxes. The outlet, resident, and inlet compressors are optional, and each or all may be omitted depending on the properties of the target gas.

Air flow 1940 entering a sorption box may be substantially identical to ambient air. The air flow may pass through a humidity filter, such as a desiccant layer, in order to decrease the air flow's moisture content. The air may pass through a compressor to reduce the air flow volume as it enters the sorption box. These measures will have the effect of reducing the pressure and increasing the concentration of the undesirable gas in the air flow 1942, which will assist the sorption process of the sorption units.

Air flow 1944 entering the piping between the sorption box and the collection container may be substantially concentrated with the undesirable gasses previously captured by the sorption units. During the desorption process, the outlet valve is opened, and the outlet (vacuum) pump and outlet compressor may be engaged, thereby reducing the air pressure and impelling the air flow, diverting the oxygen, nitrogen, carbon dioxide, previously trapped gas and air, etc., in the atmospheric air, from the air flow, and compressing the air flow 1946 as it enters the collection container.

By collecting and at least partially emptying the gas from the sorption boxes, the sorption boxes are able to regain their sorption capacity and continue remediating the ambient air. There may be two or more sorption boxes, and each sorption box may be preceded in a series (or in parallel) by a sorption box of a different function, such that each sorption box in a series (or in parallel) is dedicated to resolve a certain category of air quality problem, such as eliminating combustible air, toxic air, and/or infected air. The sorption boxes connect and convey fluid to the collection container via a configuration of (vacuum) pumps, valves, pipes, flow meters, tubes, fittings, and adapters. Control of the pumps, valves, etc., is obtained via an electrical and mechanical control system 1900. The control system may, upon receiving pressure, concentration, time, flow rate, weight change, gas type, temperature, humidity, sorption type, sorption material capacity and kinetics, compressor activity, pump activity, ventilation system activity, and other feedback parameters from various sensors indicating that the sorption box, collection container, or ambient air has reached a parameter threshold, engage the valves to open, engage the pump to pump air into the collection container, engage the compressor to reduce the air volume entering the collection container, etc. The control system may be in wired or wireless informational communication with an ambient sensor 1903, with the ambient sensor being disposed outside any given sorption box or collection container, and configured to capture data corresponding to the ambient air. The control system may calculate or predict, in advance, the occurrence and time of occurrence of various events, such as parameter thresholds, or the time required for various events, such as sorption or desorption, based on the various feedback parameters.

The controller may utilize SCADA (supervisory control and data acquisition) software.

In one variation, the control system is configured to receive weight measurements of the sorption boxes transmitted by weight sensors, and permit and facilitate fluid flow from the sorption box to the container when the weight measurement of the sorption box exceeds a designated threshold. The weight sensors may comprise a scale disposed below a given sorption box.

The control system may comprise a single central controller/control system, or a combination of a central master control controller (or system) and a series of local control controllers (or systems), with each sorption box and collection container having its own local control system 1902. Each local control system may be configured to electrically and mechanically control the local valves, pumps, compressors, ventilation systems, etc., and be in informational communication with the local sensors 1918. With a local component being a component being disposed inside or at an inlet or outlet of a sorption box or collection container to which the local control system is dedicated. Each local control system in turn may be in electrical and/or informational communication with the central master control system. Informational communication may occur through any wired or wireless protocol. In one variation, each collection container is in dedication connection to a single sorption box. In another variation, collection containers may be shared between sorption boxes. In this variation, the control system may engage the pumps, valves, etc., to prevent fluid from flowing from a first sorption box, into the collection container, and then into a second sorption box. This will prevent the duplicative process of the second sorption box being required to convey the fluid flow back into the collection container. This shared-container configuration has the advantage of a continual, uninterrupted sorption process—at least one of the sorption boxes can perform a sorption process while at least one other sorption box desorps its previously sorped gas into the collection container. In yet another variation, each sorption box is engaged with a plurality of collection containers. In yet another variation, each sorption box is engaged with a plurality of collection containers, but these collection containers are also shared with other sorption boxes.

In one embodiment, the collection container is engaged with a compressor. In one variation, the compressor may be disposed between the sorption box and the collection container, in order to compress the air received from the sorption box before conveying it into the collection container. In another variation, the compressor is disposed within the collection container and is configured to compress the air within the collection container. The compressor may be electrically and informationally engaged with the control system, which is configured to determine and set the compression power/flow rate based on various sensor-derived parameters. These sensor-derived parameters may be continually or intermittently entered into an equation to determine the most electrically and/or mechanically efficient and/or expedient compressor power/rate based on the detected or expected gas sorption and conveyance. These parameters may include, as mentioned, the fluid pressure detected within the compressor, the fluid pressure detected in the collection container, the pressure detecting at the piping and/or valves, the weight change of the sorption box, the weight change of the collection container, the category of gas detected in the sorption box, the flow rate detected between the ambient air and the sorption box, the flow rate detected between the sorption box and the collection container, and the inflation/deflation measurements of the collection container itself. If the collection container is shared between sorption boxes, then the parameters may also pertain to the other sorption boxes as well. If multiple collection containers are engaged to a given sorption box, then the parameters of each collection container is used to determine the compressor rate/power for the other collection containers.

In one variation, the compressor and/or pump may be positioned at the pass-through walls of the sorption box or in the sorption box, so that the ambient air may be compressed and impelled prior to being conveyed into the collection container.

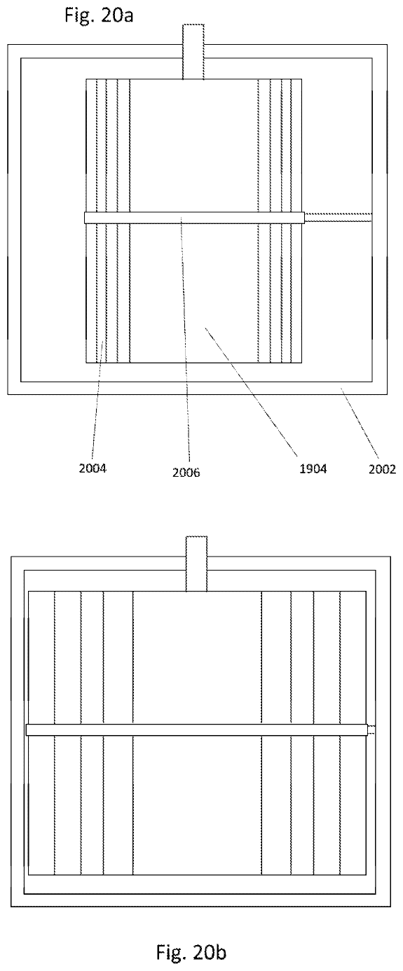

In one variation, the collection containers are fixed in their material dimensions. In another variation, the collection containers are made of flexible and/or expandable materials to enable deflation when not in use and gradual inflation during use. In a third variation, as shown in FIG. 20a-20b, the collection 1906 container is made primarily of a flexible material, and is disposed in a rigid shell 2002—once the dimensions reach those of the rigid shell, the collection container would not be expanded further.

In one embodiment, as shown in FIG. 20, the collection containers operate like diaphragms—that is, they passively expand or contract based on their internal pressure as gas is received and compressed. In an additional embodiment non-exclusive to the prior embodiment, the body of the collection containers are mechanically expanded. Mechanical expansion enables the collection container to undergo a pressure drop, thereby enabling more effective inward fluid flow as well as increased storage capacity. The mechanical expansion may occur via a series of flaps 2004 or accordion pleats which are kept flat via a mechanical ring or belt 2006. As the ring or belt is loosened, the flaps are free to open; if the flaps are attached to the ring or belt, then as the latter is mechanically opened, so too do the flaps. The ring or belt may be controlled by a pulley/wheel system, with mechanical control over a wheel upon which the belt is wound determining the length of the belt. If the belt is not attached to but merely surrounding the collection container, then the collection container expands as the amount of air contained therein increases. If the belt is attached at various points to the pleats, then as the wheel is mechanically rotated, the belt will drag the pleats further from the center of the collection container, thereby physically and actively expanding them. Mechanical control over the belt may be exercised by the control system.

In one embodiment, the control system comprises GPS technology for detecting the location of the system, if the control system is in proximity to the sorption boxes, or otherwise the sorption boxes, and in particular, the collection containers. When a collection container is detected at being at maximum capacity, which may include being in a condition of a maximum inflation, a maximum concentration, and as having maxed out all compression capacity, then the control system may, first, close all valves and conveyances out of the sorption boxes and/or into the collection containers, and second, relay a max capacity signal to a pertinent third party, such as a fire department, property manager, or dedicated waste removal organization. The third party will then be on notice to collect and replace the collection containers. In one variation, the control system sends a signal to the pertinent third party prior to the collection container being at maximum capacity, with the time difference associated with the time required for the third party to arrive at the premises to collection the collection container. The signal may be directed to a database or individual(s), and sent via an email, messaging application, or system notification/update.

In one embodiment, the control system is configured to detect a given (high) concentration of an undesirable gas in a room or closed area. When that given concentration is detected, the system sends instructions to a ventilation system, which may comprise fans and/or pumps, to begin conveying air into one or more sorption boxes. If the air remediation system detects that a given (low) concentration of the undesirable gas, the system sends instructions to the ventilation system to cease the conveyance of air into the one or more sorption boxes.

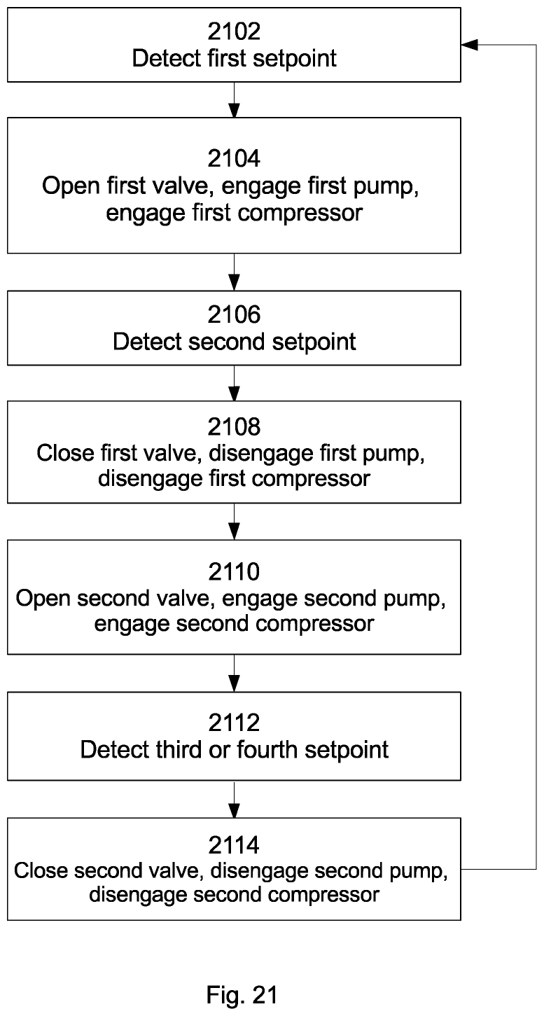

As shown in FIG. 21, the presently described event may be configured to operate an “Alternation Program”. The control system may detect a first setpoint 2102, which requires that a first sorption box needs to be desorbed of its gas into the collection container. This first setpoint maybe a first sensor detecting that the first sorption units have reached their capacity based on a direct measurement such as concentration level or an indirect measurement such as the weight of the sorption unit (or the sorption box as a whole). Alternatively, the first setpoint may be the lapsing of a given period of time since the sorption box began its sorption process. The control system may then open the first valve, engage the first pump, and engage the first compressor 2104 in order to enable airflow from the first sorption box into the collection container, decrease the pressure of the gas to impel the airflow, and then concentrate the gas in order to increase the volumetric efficiency of the collection container. The control system may then detect a second setpoint 2106, which requires that the second sorption box needs to be desorbed of its gas into the collection container, or that the first sorption box is sufficiently desorbed in order to being sorbing gas from the ambient air. This second setpoint may be any of the exemplary setpoints designated for the first setpoint, except here they are directed toward the second sorption box. The second setpoint may also be the first sensor detecting that the first sorption units have refreshed their capacity. The control system may then close the first valve, disengage the first pump, and disengage the first compressor 2108, and then open the second valve, engage the second pump, and engage the second compressor 2110. The control system may detect a third setpoint 2112, which requires that the first sorption box needs to be desorbed of its gas into the collection container, or that the second sorption box is sufficiently desorbed in order to being sorbing gas from the ambient air. This setpoint may again be any of the exemplary setpoints designated for the first setpoint, or that the second sorption units have refreshed their capacity. The system may then close the first valve, disengage the first pump, and disengage the first compressor 2114. The process may then repeat from the first step. The sorption boxes may share a compressor, vacuum pump, piping, and valves in common.

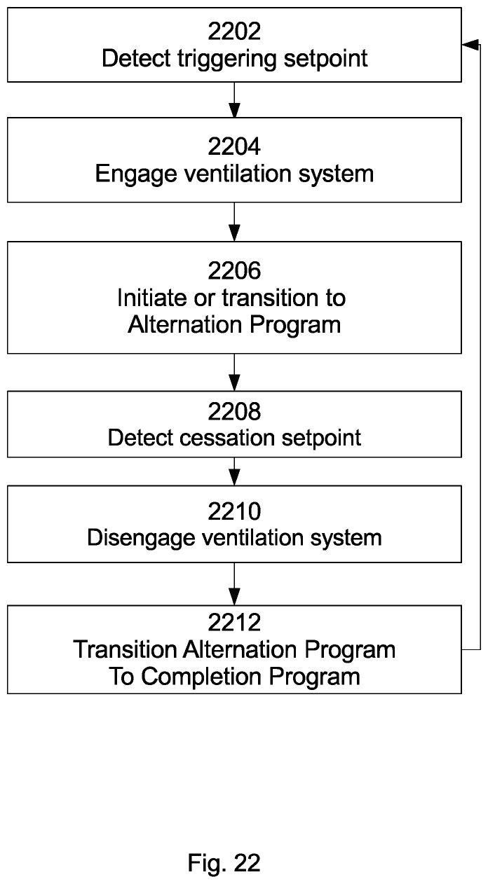

As shown in FIG. 22, the presently described device may be configured to operate a “Basic Program”. The control system may detect an initial setpoint via an ambient air sensor 2202. This sensor is configured to detect the concentration of a given target gas in the ambient air. The initial setpoint corresponds to a concentration of the target gas beyond which the operators of the device do not wish the concentration to exceed, and therefore triggers the Basic Program. The control system then engages a ventilation system of the first and/or second sorption box 2204. This engagement may occur directly, or via an intermediary of a local control system. The ventilation system may include any component or operation that enables or impels airflow into a sorption box. The control system may then initiate (or transition back into) the Alternation Program previously described 2206. The control system may then detect a cessation setpoint via the ambient air sensor 2208. The cessation setpoint corresponds to a concentration of the target gas of which the operators do not require further decrease. The control system may then disengage the ventilation system 2210 and transition the Alternation Program into a Completion Program 2212, in which the desorbing sorption box continues desorbing into the collection container until adequate desorption or refreshment of the sorption capacity is achieved, but the previously absorbing sorption box ceases absorbing from the ambient air. The process may then repeat from the first step. The ventilation system may also be disengaged, briefly, if the control system detects that the sorption boxes are at full capacity and require desorption. Alternatively, the Alternation Program may control the ventilation system by disengaging it when a sorption box is desorbing.

An “Emergency Program” may interrupt the Basic Program. The Emergency Program may be initiated if concentration of a flammable gas approaches combustion—in this case, the present device, which has electrical and/or mechanical components, could itself initiate the combustion thereof, and therefore, the Emergency Program sends a warning signal to a designated third party and then shuts down the present device.

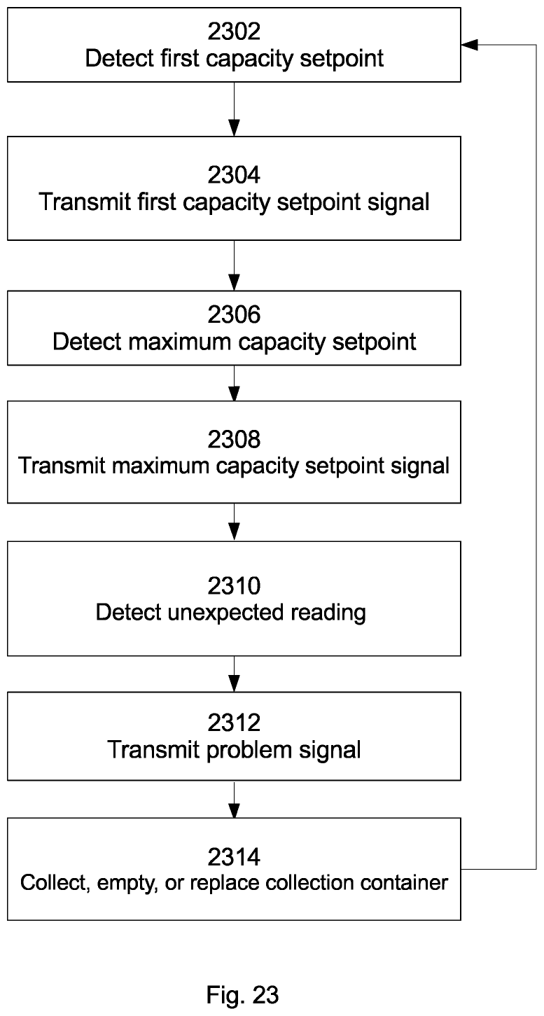

As shown in FIG. 23, the presently described device may be configured to operate a “Transmit Signal Program” concurrently with the Basic Program and/or the Alternation Program. The control system may detect a first capacity setpoint 2302. The first capacity setpoint is a concentration of target gas in a collection container which, if a third party responsible for collecting, emptying, or replacing the collection container were to leave upon receipt of a signal, would arrive when the collection container is at its maximum concentration capacity. The control system then transmits a first capacity setpoint signal to the third party 2304. If the control system detects a maximum capacity setpoint 2306, which is the maximum concentration which the collection container can or should reasonably and safely hold, it then transmits a maximum capacity setpoint signal to the third party 2308. If the control system detects an unexpected or problematic reading of any sensor, whether in the collection container or the sorption boxes 2310, then system will then transmit an inspection signal to the third party 2312 instructing the third party to inspect the present device for broken, damaged, or expired parts. An exemplary problematic reading may show a lack of decrease of a target gas concentration in a sorption box despite the operating of the ventilation system for that sorption box, which would indicate that the sorption unit of that sorption box has reached the end of its lifespan and must be replaced. The collection container is then collected, emptied, or replaced 2314, and the process repeats from the first step.

While the term “air” is used frequently throughout, and air generally contains about 78% nitrogen, 21% oxygen, and small amounts of other gases, too, it is understood that those amounts of other gases may increase, and that the air can be further contaminated with various toxic and/or flammable glasses, airborne pathogens, etc. As such, “air”, as used above, refers to air that may be in its general composition or contaminated with any gasses or particles.

In one embodiment, system pressure is measured via a pressure sensor/gauge, transmitted to the controller, and compared to a pressure setpoint controlled by the controller. Based on the comparison (and optionally, other sensor feedback data), the controller may adjust various valves or other restrictions throughout the device. The restrictions may include fixed restrictions, such as Venturo or Orifice place, or variable restrictions, such as needle valves, which may be adjusted by the controller (i.e., slightly opened or slightly closed) based on the comparison in order to bring the measured pressure toward the pressure setpoint. Restrictions may be coupled with back pressure regulators or other restrictions. Pressure sensors, measured pressure, and the pressure setpoint may be features of sub-components of the system, such as reservoir tanks coupled to compressors.

In one embodiment, the inlet ventilation system may comprise a compressor or blower (i.e., a fan and pump), which may be connected to various ducts, including a dedicated duct inlet. The compressor may have additional air tanks to pressure air and gas mixtures. The system may also feature an outlet ventilation system (i.e., column effluent), comprising vacuum pumps and/or compressors, with pumped and/or compressed air configured to be released into an atmosphere or into a specific collection bag/containers.

Various filters may be disposed throughout the ventilation systems, including filters directed toward dust, smoke, humidity (water vapor), or various molecules (via membrane filters). In one variation, humidity control is accomplished via coupling humidity sensors, humidity setpoints, humidity filters, motarized valves, and the controller to direct the flow of air having a humidity level greater than the setpoint to be filtered via the humidity filters.

The collection bags/containers may be coupled to the system for receiving contaminated or exhaust air via valves, vacuum pumps, and/or compressors. In one configuration, a vacuum pump and a compressor may each be disposed on opposite sides of the collection bag/container and system engagement. In addition, the engagement may be measured or otherwise managed via flow meters, pressure sensors, additional compressors, and other ventilation components. In one variation, the collection bags/containers contain sorption materials.

In one embodiment, the system comprises a leak detection program to check the system for leaks. During this program, the various valves are opened and closed, with pressure sensors configured to detect pressure (and changes thereto) to determine whether there is any pressure lost with valves closed, etc. Determination of leaks result in notifications to relevant parties.

1. A gas control device comprising a sorption box, sorption units, a ventilation system, a sensor system, and a control system;
the ventilation system being in electrical communication with the control system, the control system being in informational communication with the sensor system;
the sorption box being an enclosure;
the sorption units made of material capable of adsorbing or absorbing gases;
the sensor system comprising one or more gas sensors configured to detect gases and transmit gas detection signals to the control system;
the ventilation system oriented to suck air through or toward the sorption box;
the control system comprising a processor programmed to receive gas detection signals from the sensor system and control ventilation.

2. The device in claim 1, additionally comprising a sorption chamber, the sorption chamber configured to hold the sorption units in place while permitting airflow from the cavity into the sorption units.

3. The device in claim 2, with the sorption chamber being attached to a wall of the sorption box.

4. The device in claim 2, with the ventilation system configured to transfer airflow from the cavity into the sorption units via ducts.

5. The device in claim 1, the sorption box being an enclosure against an atmosphere surrounding the sorption box.

6. The device in claim 1, the sorption box comprising passage walls, the passage walls comprising one or more sorption openings, the one or more sorption openings providing access into the cavity.

7. The device in claim 1, the sorption box configured to connect to tubing, piping, vents, or other HVAC components.

8. The device in claim 1, also comprising one or more dust filters, the one or more dust filters configured to filter particles smaller than 1 mm in diameter contained within the airflow suctioned by the ventilation system.

9. The device in claim 1, with the one or more gas sensors disposed inside the sorption box and configured to detect contaminant gas levels inside the sorption box and transmit internal contaminant gas signals to the control system.

10. The device in claim 9, the control system configured to receive contaminant gas signals from the sensor system and control the one or more fans based on the contaminant gas signals.

11. The device in claim 1, the one or more gas sensors configured to detect a first threshold of gas contaminants and a second threshold of gas contaminants, with the first threshold of gas being less dense than the second threshold of gas; and upon detecting the first threshold of gas contaminants, transmit a first contaminant threshold signal to the control system, and upon detecting the second threshold of gas contaminants, transmit a second contaminant threshold signal to the control system; the control system configured to receive a first and second contaminant threshold signals from the sensor system.

12. The device in claim 1, with the ventilation system comprising one or more fans, with the one or more fans configured to suck air from the cavity and blow air out of the sorption box.

13. The device in claim 1, with the ventilation system comprising one or more fans, with the one or more fans configured to suck air into the sorption box.

14. The device in claim 12, the control system configured to determine if the contaminant gas levels are too high using the contaminant gas signals, and if so, increase rotation speeds of one or more fans in an inlet orientation.

15. The device in claim 1, the control system configured to record gas detection data.

16. The device in claim 1, the control system configured to send gas detection data to a mobile device.

17. A gas control device comprising a sorption box, sorption units, a ventilation system, and a sensor system;
the sorption box being an enclosure;
the sorption units made of material capable of neutralizing gases;
the sensor system comprising one or more gas sensors configured to detect gases and transmit gas detection signals to the control system;
the ventilation system oriented to blow air out of or into the sorption box.

18. The device in claim 17, additionally comprising a sorption chamber, the sorption chamber configured to hold the sorption units in place while permitting airflow from the cavity into the sorption units.

19. The device in claim 2, with the ventilation system configured to transfer airflow from the cavity into the sorption units via ducts.

20. A gas control device comprising a sorption box, a sorption chamber, a ventilation system, and a sensor system;
the sorption box being an enclosure;
the sorption chamber containing material capable of neutralizing gases;
the sensor system comprising one or more gas sensors configured to detect gases and transmit gas detection signals;
the ventilation system oriented to blow air out of the sorption box based on the gas detection signals.
